(19) United States
(12) Patent Application Publication
(10) Pub. No.: US 2012/0095110 A1
(43) Pub. Date: Apr. 19, 2012

(54) BRANCHED POLYMER DISPERSANTS

(75) Inventors: Paul Hugh Findlay, Liverpood (GB); Brodyck James Lachian Royles, Liverpood (GB); Roselyne Marie Andree Baudry, Liverpood (GB); Neil John Simpson, Liverpood (GB); Sharon Todd, Liverpood (GB); Steven Paul Rannard, Liverpood (GB)

(73) Assignee: Unilever PLC, London (GB)

(21) Appl. No.: 13/379,639

(22) PCT Filed: Jun. 22, 2010
(86) PCT No.: PCT/GB2010/001214

§ 371 Date: Dec. 20, 2011

(30) Foreign Application Priority Data
Jun. 22, 2009 (GB) 0910750.9

(51) Int. Cl. A61K 47/32; C08L 47/00; C09D 7/02; A23L 1/226; A01N 25/22; C10M 149/10; A61K 8/81; C10M 145/14; C08F 236/20; C04B 24/26

(52) U.S. Cl. 514/772.5; 526/263; 526/323.2; 524/548; 524/559; 524/5; 514/772.6; 508/264; 512/1; 508/472; 426/650

(57) ABSTRACT

The present invention relates to the use of a branched addition copolymer as a dispersant in a gaseous, liquid or solid formulation in a range of applications and to the copolymers per se wherein the copolymer is obtainable by an addition polymerisation process, wherein said copolymer comprises: at least two chains which are covalently linked by a bridge other than at their ends; and wherein the at least two chains comprise at least one ethylenically monounsaturated monomer, and wherein the bridge comprises at least one ethylenically polyunsaturated monomer; and wherein the polymer comprises a residue of a chain transfer agent and wherein the mole ratio of polyunsaturated monomer(s) to monounsaturated monomer(s) is in a range of from 1:100 to 1:4; and wherein the branched copolymer dispersant contains anchoring, solubilising or stabilising moieties and wherein the resulting copolymer has a weight average molecular weight of less than 100,000 Da.

### CROSS-REFERENCE TO RELATED APPLICATIONS

This application is the national phase entry of PCT Application No. PCT/GB2010/00121.4, filed Jun. 22, 2010, which claims priority to GB Application No. 0910750.9, filed Jun. 22, 2009. The disclosures of said applications are hereby incorporated herein by reference.

### BACKGROUND

1. Technical Field

The present invention relates to branched addition copolymers. More specifically, the present invention relates to compositions of branched addition copolymers having a weight average molecular weight of less than 100,000 Da and their use as dispersing agents, methods for the preparation of such copolymers, formulations comprising the branched addition copolymers and the use of the formulations as dispersants. When the copolymers are used as dispersants they are effective at low doses in formulations. In addition, in solution, these formulations exhibit low solution viscosities, the formulations can be formed at high dispersed phase content; the formulations can be used to treat unmodified pigments and can also reduce milling times resulting in smaller particle sizes.

2. Background of the Invention

Polymer dispersants. Dispersants are usually used to stabilise an immiscible or insoluble particle in a bulk medium. The bulk medium can be solid, liquid or gaseous in nature. The dispersant acts to prevent aggregation of the particles in the bulk phase. In addition, dispersants usually reduce any increase in viscosity of a dispersion or colloid. This is achieved by aggregation of the particles. Increasingly dispersants are polymeric in nature and typically posses units to anchor them onto the insoluble or immiscible particle while other moieties act as solubilising or stabilising units by interaction with the bulk medium or through particle-particle repulsion, such as via electrostatic mechanisms; occasionally the same unit can provide all of these properties.

Block or graft copolymers are particularly useful in this respect as the distinct structures within the polymers can behave as anchoring, solubilising or stabilising units, strongly interacting with the particle and the hulk phase separately.

Amphiphilic copolymers maybe used as dispersants of particulates in aqueous media where the hydrophobic portions of the polymer adsorb onto the particle surface while the hydrophilic groups, typically charged units such as carboxylic acids, aid the stabilisation via particle-particle repulsion and strong solvent interaction.

WO 2006/042033 A2 (Flink ink) discloses a method for preparing ink binders, which contains branched vinyl polymers, in a medium that includes a non-volatile polyol-based fatty oil. The branched polymers described therein were prepared with at least one monomer having at least two ethylenically unsaturated polymerisable groups per molecule, preferably divinyl benzene (DVB), added at between 1.5 and 3.25% w/w (of the total weight of monomers polymerised); at least one aliphatic ethylenically unsaturated monomer added at between 20 to 25% w/w (of the total weight of monomers polymerised) and at least one aromatic monomer, preferably styrene, at between 60 to 70% w/w (of the total weight of monomers polymerised) and thereafter reacting said mixture in a free-radical polymerization reaction using a semi-batch process to form a copolymer, wherein the molecular weight of the branched polymer is preferably in the range 1000 to 10,000 Da and preferably with a Tg of 70° C.

WO 2000/037542 (3M) describes a method for preparing dendritic polymer dispersants for the dispersing of hydrophobic particles comprising a derivitised dendritic polymer having at least one peripheral ionisable moiety and at least one peripheral non-polymeric hydrocarbon hydrophobic moiety. The dendritic dispersant was prepared using: a commercially available 3^{rd} or 5^{th} generation polyol (Boltorn H30 or H50, respectively), incorporating hydrophobic segments via the reaction with fatty acids, preferably containing between 8 to 22 carbons, such as via esterification with stearic acid, or through the incorporation of hydrophilic segments, such as through the reaction with succinic anhydride. The derivatized dendritic polymers have a preferable molecular weight of 15,000 to 35,000 Da.

WO2008/03037612 (CIBA) relates to a liquid dispersant based on polar polyamines, or modified polycarboxylic acids characterized by a “dendritic” structure. Here the terminus of the polymer is modified by a diol-containing carboxylic acid which itself is further modified with a fatty acid unit. The dendritic polymer dispersant is synthesised via a convergent or divergent synthetic route.

WO2007/135032 (BASF) discloses the use of highly branched polycarbonate-based polymeric pigment dispersants. The hydroxyl termini of the polymers are functionalised with aliphatic or aromatic hydrophobic groups containing between 1 to 20 carbon atoms.

US2004/0097685 (Neil and Weinkauf) discloses the use of hyperbranched polyurethane dispersants containing between 2 to 100 residual isocyanate units and having a molecular weight of between 500 to 50,000 Da and reacting the subsequent polyisocyanate with an alkyl-functional polyalkylene oxide, where the alkyl group contains between 3 to 40 carbon atoms.

WO2007/110333 (CIBA) discloses the synthesis of a functionalised poly(ethylene imine) (PEI)-based polymer dispersant via the grafting of hydrophobised alkylene oxide units onto the branched polymer backbone. These units posses alkylene carboxylic units of between 1 to 22 carbon atoms.

WO98/18839 (Du Pont) discloses the use of a branched polymer dispersing agent in an aqueous formulation. The branched polymer dispersant is amphiphilic in nature having a molecular weight in the range of 5.000 to 100,000 Da containing both hydrophilic and hydrophobic sections containing at least 10% by weight of carboxylic units. The branched polymers are prepared in a two-step process via the use of a catalytic chain transfer agent in the first step to prepare functional macromonomers with terminal vinyl groups that are utilised in the second stage of the preparation.

US 2006/0106133 A1 discloses an ink-jet ink comprising an amphiphilic polymer, wherein the polymer comprises hydrophilic and hydrophobic portions, at a molecular weight range from 300 to 100,000 Daltons, and may be in the form of a straight chain polymer, a star-form polymer or an emulsion form having a polymer core. A chain transfer agent is not used in the production of the polymer. The polymer is used as a wetting aid in the formation of uniform ink droplets on the substrate.

Branched Polymers. Branched polymers are polymer molecules of a finite size which are branched. Branched polymers differ from cross-linked polymer networks which tend towards an infinite size having interconnected molecules and which are generally not soluble. It has now been found by the inventors that in some instances, branched polymers have advantageous properties when compared to analogous linear polymers. For instance, it has been reported that solutions of branched polymers are normally less viscous than solutions of analogous linear polymers. Moreover, higher molecular weights of branched copolymers can be solubilised more easily than those of corresponding linear polymers. In addition, as branched polymers tend to have more end groups than a linear polymer the branched polymers generally exhibit strong surface-modification properties.

Branched polymers are usually prepared via a step-growth mechanism via the polycondensation of suitable monomers and are usually limited via the chemical functionality of the resulting polymer and the molecular weight. In addition polymerisation, a one-step process can be employed in which a multifunctional monomer is used to provide functionality in the polymer chain from which polymer branches may grow. However, a limitation on the use of a conventional one-step process is that the amount of multifunctional monomer must be carefully controlled, usually to substantially less than 0.5% w/w in order to avoid extensive cross-linking of the polymer and the formation of insoluble gels. It is difficult to avoid cross-linking using this method, especially in the absence of a solvent as a diluent and/or at high conversion of monomer to polymer.

WO 99/46301 discloses a method of preparing a branched polymer comprising the steps of mixing together a monofunctional vinylic monomer with from 0.3 to 100% w/w (of the weight of the monofunctional monomer) of a multifunctional vinylic monomer and from 0.0001 to 50% w/w (of the weight of the monofunctional monomer) of a chain transfer agent and optionally a free-radical polymerisation initiator and thereafter reacting said mixture to form a copolymer. The examples of WO 99/46301 describe the preparation or primarily hydrophobic polymers and, in particular, polymers wherein methyl methacrylate constitutes the monofunctional monomer. These polymers are useful as components in reducing the melt viscosity of linear poly(methyl methacrylate) in the production of moulding resins.

WO 99/46310 discloses a method of preparing a (meth)acrylate functionalised polymer comprising the steps of mixing together a monofunctional vinylic monomer with from 0.3 to 100% w/w (based on monofunctional monomer) of a polyfunctional vinylic monomer and from 0.0001 to 50% w/w of a chain transfer agent, reacting said mixture to form a polymer and terminating the polymerisation reaction before 99% conversion. The resulting polymers are useful as components of surface coatings and inks, as moulding resins or in curable compounds, for example curable moulding resins or photoresists.

WO 02/34793 discloses a rheology modifying copolymer composition containing a branched copolymer of an unsaturated carboxylic acid, a hydrophobic monomer, a hydrophobic chain transfer agent, a cross-linking agent, and, optionally, a steric stabilizer. The copolymer provides increased viscosity in aqueous electrolyte-containing environments at elevated pH. The method for production is a solution polymerisation process. The polymer is lightly cross-linked, less than 0.25%.

U.S. Pat. No. 6,020,291 discloses aqueous metal working fluids used as lubricants in metal cutting operations. The fluids contain a mist-suppressing branched copolymer, including hydrophobic and hydrophilic monomers, and optionally a monomer comprising two or more ethylenically unsaturated bonds. Optionally, the metal working fluid may be an oil-in-water emulsion. The polymers are based on polyacrylamides) containing sulfonate-containing and hydrophobically modified monomers. The polymers are cross-linked to a very small extent by using very low amount of bis-acrylamide, without using a chain transfer agent.

### SUMMARY

Herein disclosed is a method of using of a brandied addition copolymer as a dispersant in a gaseous, liquid or solid formulation wherein the copolymer is obtainable by an addition polymerisation process, wherein said copolymer comprises: at least two chains which are covalently linked by a bridge other than at their ends; and wherein the at least two chains comprise at least one ethylenically monounsaturated monomer, and wherein the bridge comprises at least one ethylenically polyunsaturated monomer; and wherein the polymer comprises a residue of a chain transfer agent; and wherein the mole ratio of polyunsaturated monomer(s) to monounsaturated monomer(s) is in a range of from 1:100 to 1:4; and wherein the branched copolymer dispersant contains anchoring, solubilising or stabilising moieties and wherein the resulting copolymer has a weight average molecular weight of less than 100,000 Da.

In an embodiment, the residue of the chain transfer agents comprises 0.05 to 80 mole % of the copolymer based on the number of moles of monofunctional monomer. In an embodiment, the residue of the chain transfer agents comprises 0.05 to 30 mole % of the copolymer based on the number of moles of monofunctional monomer. In an embodiment, the residue of the initiator comprises 0 to 10% w/w of the copolymer based on the total weight of the monomers. In an embodiment, the residue of the initiator comprises 0.001 to 5% w/w of the copolymer based on the total weight of the monomers. In an embodiment, the residue of the initiator comprises 0.001 to 3% w/w of the copolymer based on the total weight of the monomers.

In an embodiment, the monofunctional monomer is selected from the group consisting of vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives of the aforementioned compounds as well as corresponding allyl variants thereof.

In an embodiment, the multifunctional monomer or brancher is selected from the group consisting of divinyl aryl monomers such as divinyl benzene; (meth)acrylate diesters such as ethylene glycol di(meth)acrylate, propyleneglycol di(meth)acrylate and 1,3-butylenedi(meth)acrylate; polyalkylene oxide di(meth)acrylates such as tetraethyleneglycol di(meth)acrylate, polyethyleneglycol)di(meth)acrylate and poly(propyleneglycol) di(meth)acrylate; divinyl(meth)acrylamides such as methylene bisacrylamide; silicone-containing divinyl esters or amides such as (meth)acryloxypropyl-terminated poly(dimethylsiloxane); divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate. Further examples include vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene)

In an embodiment, at least one of the monounsaturated monomer(s) and polyunsaturated monomer(s) and chain transfer agent(s) is a hydrophilic residue; and at least one of one of the monounsaturated monomer(s) and polyunsaturated monomer(s) and chain transfer agent(s) is a hydrophobic residue.

Further disclosed herein is a composition of a branched addition copolymer and its uses and applications thereof.

These and other embodiments, features and advantages will be apparent in the following detailed description

### DETAILED DESCRIPTION

Dispersing agents, and in particular polymeric dispersing agents, are used to stabilise particles in a hulk or continuous medium. These particles are typically insoluble or immiscible in the continuous phase and tend to range in size from sub micron to a few millimeters. Typically the particles are solid, insoluble species in the range from a few nanometers to a few microns. Increasing the size of the dispersed particles leads to aggregation, and flocculation in the dispersed phase, this is particularly true for crystalline materials or particles with highly associating groups. It is generally required that the dispersed particles are distributed evenly within the bulk phase and to this end a dispersing aid is required.

The bulk phase can be gaseous, liquid, or solid in nature. Commonly the bulk phase is a liquid, resulting in a colloidal suspension of particles where the dispersant is either fully or partially dissolved in the bulk phase. The bulk phase can also be gaseous, giving rise to particulate aerosols of solids, such as in a smoke. The bulk phase can also be solid in nature where a solid particle is dispersed in a bulk solid phase, usually prior to some further processing step, such as in powder coatings.

To be effective the dispersant must posses three key functional groups, namely:

An Anchoring Group: Which interacts with the particulate to be dispersed via surface adsorption such as through van der Waals interactions—common in the dispersion of hydrophobic materials in an aqueous medium, π-π stacking—often used with hydrophobic pigments, electrostatic interaction—where an oppositely charged dispersant is used with the particulate, via H-bonding—common with natural proteinaceous, or carbohydrate-based dispersants or via the formation of a covalent bond with the particle.

A Solvating Group: Which interacts with the dispersed phase, usually a liquid. Here the dispersant must posses a moiety which can interact with the solution or bulk phase and essentially lead to solvation of the particle. For the dispersion of hydrophobic particles in an aqueous solution these solvating units tend to be composed of oligomeric water-soluble groups. In solid-solid dispersions or solid-gas dispersions the effect of a solvating group is generally less.

A Stabilising Group: Once anchored and solvated the dispersant must reduce particle-particle interaction thereby reducing the likelihood of aggregation and ultimately precipitation of the particle. In aqueous systems this is usually achieved via the incorporation of charged species resulting in electrostatic repulsion. The solvating group can achieve this function since when well solvated it can give rise to a swollen polymer corona surrounding the particle thereby reducing the particle-particle interaction.

Commonly, different chemical groups are chosen to perform these roles within a polymeric dispersant although when chosen correctly the same unit can perform multiple functions.

It is generally required that the dispersant is at least miscible, if not completely soluble within the bulk phase, although in the case of amphiphilic dispersants this can be achieved through the use of a co-solvent of by tuning the solution pH.

Due to their large size and multiple anchoring, solvating and stabilising units polymers and especially those with a block or graft (comb) structure are particularly effective dispersants. Block or graft polymers can be engineered in such a way as to have discrete anchoring, solubilising or stabilising regions throughout their structure leading to a maximising of these properties.

Block copolymers can be formed through the reaction of two or more pre-formed oligomeric species either through a step-growth procedure, such as in the ring-opening of ε-caprolactone, or through the living addition polymerisation of vinylic monomers. Commonly, block copolymers are prepared via sequential addition of the monomer species through a step-growth or living polymerisation procedure, such as anionic polymerisation.

Graft or comb copolymers are prepared via the sequential addition of main chain monomer(s) in conjunction with a preformed macromonomer or via the grafting of a pre-formed oligomer onto a pre-formed polymer. As in the case for block copolymers the polymerisation can be via either step-growth or addition in nature.

Although both grail and comb polymers can be used effectively as dispersants they tend to be limited via their molecular weight. Additionally, the synthesis of either of these materials can be multi-step or use expensive monomers or reagents. Solubility problems can also arise where the different segments in these polymers are particularly large, especially where they can crystallise or strongly interact in their solid form.

Branched polymer dispersants can also be prepared and used effectively as dispersants although the most common form of preparing these materials is again through a multi-step process, most commonly step-growth polymerisation. Numerous examples of these polymers can be found, many are based upon the commercial material polyethylene imine) where this inherently branched polymer is further reacted with long chain hydrophilic, hydrophobic or amphiphilic groups depending upon the end use. Once again this synthetic route is multi-step and in many cases involves purification or at the very least isolation procedures.

Further, reactive backbones can also be prepared using an ABx step growth polymerisation procedure. Here the monomer has multi-functionality as it can react with multiples of itself; one monomer can react with at least two further monomers and so on, usually via a condensation reaction such as an esterification, for example the monomer possesses one carboxyl and two hydroxyl groups. Again polymers of this type are limited via their monomer classes, which tend to be expensive, and in order to provide efficient anchoring, solubilising or stabilisation the dispersant requires further chemical modification.

Branched addition copolymer dispersants have an advantage in that they can be prepared via a ‘one-pot’ procedure utilising a multitude of commercial monomers and chain transfer agents. The chemistry can thus be tuned to the specific requirements of the dispersant while maximising the surface interaction through their large size and multiple anchoring points. Graft-like structures can also be prepared utilising vinylic macromonomers in the polymerisation process while the end-termini of the polymers can be controlled through choice of chain transfer went to give almost block-like properties.

The branched copolymer dispersants of the present invention are branched, non-cross-linked addition polymers and include statistical, block, graft, gradient and alternating branched copolymers having a weight average molecular weigh of less than 100,000 Da. The copolymers of the present invention comprise at least two chains which are covalently linked by a bridge other than at their ends, that is, a sample of said copolymer comprises on average at least two chains which are covalently linked by a bridge other than at their ends. When a sample of the copolymer is made there may be accidentally some polymer molecules that are un-branched, which is inherent to the production method (addition polymerisation process). For the same reason, a small quantity of the polymer will not have a chain transfer agent (CTA) on the chain end. These dispersants can be used at low levels, have a high degree of solubility with strong particle interactions and give rise to dispersions, of low solution viscosity. The dispersants Can also be used at low dose levels leading to the possibility of high dispersed phase formulations being formed.

Additionally branched addition polymer dispersants can lead to reduced processing and milling times when used to stabilise solid particulates in liquid formulations such as dispersing pigments particles in a solvent.

The use of branched addition polymers with a weight average molecular weight less than 100 K Da allows low viscosity formulations to be prepared due to the inherent low solution viscosities of the polymer dispersant solutions arising from their branched architecture.

The branched architecture of the dispersant materials described has enhanced performance when compared to an analogous linear material and can be used at lower levels and give dispersed solutions with lower viscosities.

In addition it has been found that as dispersions formed using branched addition polymers have a higher dispersed phase concentration for comparable or lower viscosities when compared to linear dispersion systems, this can lead to higher pigment strengths and greater application speeds when utilised for pigment formulations.

Thus, it has now been found that the branched addition copolymers of the present invention are useful components of many compositions and are therefore utilised in a variety of dispersant applications.

The dispersants or dispersant formulations of the present invention can therefore be applied to the following technology areas:

Applications. In the dispersion of pigments: including organic, inorganic, metallic, pearlescent, surface treated and untreated pigments in the preparation of: inks, paints, sealants, timers, powder coating and injection moulding.

In the dispersion of metal salts including for example the inhibition of inorganic fouling, the recirculation of cooling water, anti-scaling, distillation, boiler water, oilfield fluids, oil lubrication additives (oil “detergents”), and in building materials such as for example cement and gypsum.

In the dispersion of metal particles including for example cutting and milling fluids, oil lubricants, metallic coatings, powder coatings and primers and mineral processing.

In the dispersion of organic “actives” such as for example in the pharmaceuticals/agrochemicals/biocides industries and in the food industry for food colourants, flavourings, fragrances, and also in cosmetics and sun-care products.

The dispersants or dispersant formulations can also be used in the dispersion of organisms for example to prevent biofouling.

Therefore according to a first aspect of the present invention there is provided the use of a branched addition copolymer as a dispersant in a gaseous, liquid or solid formulation wherein the copolymer is obtainable by an addition polymerisation process, wherein said copolymer comprises:

at least two chains which are covalently linked by a bridge other than at their ends; and wherein the at least two chains comprise at least one ethylenically monounsaturated monomer, and wherein the bridge comprises at least one ethylenically polyunsaturated monomer; and wherein

the polymer comprises a residue of a chain transfer agent and wherein

the mole ratio of polyunsaturated monomer(s) to monounsaturated monomer(s) is in a range of from 1:100 to 1:4; and wherein

the branched copolymer dispersant contains anchoring, solubilising or stabilising moieties and wherein the resulting copolymer has a weight average molecular weight of less than 100,000 Da.

The branched copolymer according to a first aspect of the present invention can be used as a dispersant to stabilise solid particles within a liquid phase to form a stable dispersion, or the branched copolymer dispersant can be used to stabilise solid particles within a solid phase to form a stable dispersion. Alternatively, the branched copolymer dispersant can be used to stabilise solid particles within a gaseous phase to form a stable dispersion.

The solid particles to be stabilised may be particles in a hydrophobic or hydrophilic liquid.

The branched copolymer according to the first aspect of the present invention has a weight average molecular weight of 2000 Da to 1,000,000 Da. More preferably, the branched copolymer has a weight average molecular weight of 2,000 Da to 50,000 Da. Even more preferably 5000 Da to 50,000 Da.

The branched copolymer according to the first aspect of the present invention can be used in a range of applications. For example, the branched copolymer can be used as dispersants for pigments, wherein the pigments can include organic, inorganic, metallic, and pearlescent pigments. In addition, the branched copolymer can be used as dispersants for inks, paints, sealants, tinters, powder coatings, and injection moulding applications.

The branched copolymer according to the first aspect of the present invention can also be used as dispersants for metal salts and metallic particles. For example, such applications can include the use in systems that inhibit inorganic fouling, the recirculation of cooling water, anti-scaling applications and distillation and boiler water.

In addition, the branched copolymer according to the first aspect of the present invention can also be used as dispersants for cement and/or powder coatings, for example gypsum.

Furthermore, the branched copolymer according to the first aspect of the present invention can also be used as dispersants for lubricating media, for example in oilfield fluids and oil lubrication additives (oil “detergents”).

Likewise, the branched copolymer according to the first aspect of the present invention can be used as dispersants for organic actives, such as for example actives compounds in the technology areas of pharmaceuticals, agrochemicals, biocides, food colourants, flavourings and fragrances and also as dispersants for organisms in which it is required to prevent biofouling.

The branched copolymer according to the first aspect of the present invention is preferably used as a dispersant such that the ratio of the dispersed phase to polymer is in the range of 0.1:1 to 1000:1. More preferably the polymer is applied to a dispersion the ratio of the dispersed phase to polymer is in the range of 0.1:1 to 500:1. Most preferably the polymer is applied to a dispersion the ratio of the dispersed phase to polymer is in the range of 0.2:1 to 200:1.

The branched addition copolymers of the present invention preferably comprise less than 10% by weight of impurity which may be for example in the form of unreacted reagents. More preferably, the branched addition copolymers of the present invention comprise less than 5% by weight of impurity. Even more preferably, the branched addition copolymers of the present invention comprise less than 5% by weight of impurity. Most preferably however, the branched addition copolymers of the present invention comprise less than 1% by weight of impurity in the form of total unreacted monomers and chain transfer agent.

The branched copolymer dispersants of the present invention are branched, non-cross-linked addition polymers and include statistical, block, graft, gradient and alternating branched copolymers having a weight average molecular weight of less than 100,000 Da. The copolymers of the present invention comprise at least two chains which are covalently linked by a bridge other than at their ends, that is, a sample of said copolymer comprises on average at least two chains which are covalently linked by a bridge other than at their ends. When a sample of the copolymer is made there may be accidentally some polymer molecules that are un-branched, which is inherent to the production method (addition polymerisation process). For the same reason, a small quantity of the polymer might not have a chain transfer agent (CTA) on the chain end. These dispersants can be used at low levels, have a high degree of solubility with strong particle interactions and give rise to dispersions of low solution viscosity.

Therefore according to a second aspect of the present invention there is provided improved branched addition copolymers for use as dispersants in a gaseous, liquid or solid formulation according to the first aspect of the present invention wherein the copolymer is obtainable by an addition polymerisation process, wherein said copolymer comprises:

at least two chains which are covalently linked by a bridge other than at their ends; and wherein the at least two chains comprise at least one ethylenically monounsaturated monomer; and wherein

the bridge comprises at least one ethylenically polyunsaturated monomer; and wherein

the polymer comprises a residue of a chain transfer agent; and wherein

the mole ratio of polyunsaturated monomer(s) to monounsaturated monomer(s) is in a range of from 1:100 to 1:4; and wherein

the branched copolymer dispersant contains anchoring, solubilising or stabilising moieties and wherein the resulting copolymer has a weight average molecular weight of less than 100,000 Da.

When preparing a branched addition copolymer according to the present invention, a chain transfer agent is employed. The chain transfer agent (CIA) is a molecule which is known to reduce molecular weight during a free-radical polymerisation via a chain transfer mechanism. The amphiphilicity, emulsion stabilising power, responsive nature and susceptibility to controlled demulsification can be controlled through the choice of chain transfer agent. These agents may be any thiol-containing molecule and can be either monofunctional or multifunctional. The agent may be hydrophilic, hydrophobic, amphiphilic, anionic, cationic, neutral, zwitterionic or responsive. The molecule can also be an oligomer or a pre-formed polymer containing a thiol moiety. (The agent may also be a hindered alcohol or similar free-radical stabiliser). Catalytic chain transfer agents such as those based on transition metal complexes such as cobalt bis(borondifluorodimethyl-glyoximate) (CoBF) may also be used. Suitable thiols include but are not limited to C₂ to C₁₈ branched or linear alkyl thiols such as dodecane thiol, functional thiol compounds such as thioglycolic acid, thio propionic acid, thioglycerol, cysteine and cysteamine. Thiol, containing oligomers or polymers may also be used such as for example poly(cysteine) or an oligomer or polymer which has been post-functionalised to give a thiol group(s), such as polyethyleneglycol) (di)thio glycollate, or a pre-formed polymer functionalised with a thiol group. For example, the reaction of an end or side-functionalised alcohol such as poly(propylene glycol) with thiobutyrolactone, to give the corresponding thiol-functionalised chain-extended polymer. Multifunctional thiols may also be prepared by the reduction of a xanthate, dithioester or trithiocarbonate end-functionalised polymer prepared via a Reversible Addition Fragmentation Transfer (RAFT) or Macromolecular Design by the Interchange of Xanthates (MADIX) living radical method. Xanthates, dithioesters, and dithiocarbonates may also be used, such as cumyl phenyldithioacetate. Alternative chain transfer agents may be any species known to limit the molecular weight in a free-radical addition polymerisation including alkyl halides and transition metal salts or complexes. More than one chain transfer agent may be used in combination.

Hydrophobic CTAs include but are not limited to linear and branched alkyl and aryl (di)thiols such as dodecanethiol, octadecyl mercaptan, 2-methyl-1-butanethiol and 1,9-nonanedithiol. Hydrophobic macro-CTAs (where the molecular weight of the CIA is at least 1000 Daltons) can be prepared from hydrophobic polymers synthesised by RAFT (or MADIX) followed by reduction of the chain end, or alternatively the terminal hydroxyl group of a preformed hydrophobic polymer can be post functionalised with a compound such as thiobutyrolactone.

Hydrophilic CTAs typically contain hydrogen bonding and/or permanent or transient charges. Hydrophilic CTAs include but are not limited to: thio-acids such as thioglycolic acid and cysteine, thioamines such as cysteamine and thio-alcohols such as 2-mercaptoethanol, thioglycerol and ethylene glycol mono- (and di-)thio glycollate. Hydrophilic macro-CTAs (where the molecular weight of the CTA is at least 1000 Daltons) can be prepared from hydrophilic polymers synthesised by RAFT (or MADIX) followed by reduction of the chain end, or alternatively the terminal hydroxyl group of a preformed hydrophilic polymer can be post functionalised with a compound such as thiobutyrolactone.

Amphiphilic CTAs can also be incorporated in the polymerisation mixture, these materials are typically hydrophobic alkyl-containing thiols possessing a hydrophilic function such as but not limited to a carboxylic acid group. Molecules of this type include mercapto undecylenic acid.

Responsive macro-CTAs (where the molecular weight of the CTA is at least 1000 Daltons) can be prepared from responsive polymers synthesised by RAFT (or MADIX) followed by reduction of the chain end, or alternatively the terminal hydroxyl group of a preformed responsive polymer, such as poly(propylene glycol), can be post functionalised with a compound such as thiobutyrolactone.

The residue of the chain transfer agent may comprise 0 to 80 mole % of the copolymer (based on the number of moles of monofunctional monomer). More preferably the residue of the chain transfer agent comprises 0 to 50 mole %, even more preferably 0 to 40 mole % of the copolymer (based on the number of moles of monofunctional monomer). However, most especially the chain transfer agent comprises 0.05 to 30 mole %, of the copolymer (based on the number of moles of monofunctional monomer). The dispersing power of the polymer can be controlled through the choice of CTA, as these residues, where present, can act as anchoring, solubilising or stabilising groups.

Preferred chain transfer agents include linear and branched alkyl and aryl(di)thiols such as n-dodecanethiol, t-dodecanethiol, octadecyl mercaptan, 2-methyl-1-butanethiol and 1,9-nonanedithiol. Hydrophilic CTAs including thio-acids such as thioglycolic acid and cysteine, thioamines such as cysteamine and thio-alcohols such as 2-mercaptoethanol, thioglycerol and ethylene glycol mono- (and di-)thio glycollate mercapto propionic acid and mercapto propylsulfonate.

The initiator is a free-radical initiator and can be any molecule known to initiate free-radical polymerisation such as for example azo-containing molecules, persulfates, redox initiators, peroxides and benzyl ketones. These may be activated via thermal, photolytic or chemical means. Examples of these include but are not limited to: 2,2′-azobisisobutyronitrile (AIBN), azobis(4-cyanovaleric acid), benzoyl peroxide, diisopropyl peroxide, cumylperoxide, 1-hydroxycyclohexyl phenyl ketone, hydrogenperoxide/ascorbic acid. Iniferters such as benzyl-N,N-diethyldithiocarbamate can also be used. In some cases, more than one initiator may be used. The initiator may be a macroinitiator having a molecular weight of at least 1000 Daltons. In this case, the macroinitiator may be hydrophilic, hydrophobic, or responsive in nature. The dispersing power of the polymer can be controlled through the choice of initiator, especially in the case where macromolecular pseudo living radical initiators are utilised, as these residues, where present, can also act as anchoring, solubilising or stabilising groups.

Preferably, the residue of the initiator in a free-radical polymerisation comprises from 0 to 10% w/w of the copolymer based on the total weight of the monomers. More preferably 0.001 to 8% w/w of the copolymer, and especially 0.001 to 5% w/w, of the copolymer based on the total weight of the monomers.

It is also preferred that the residual material or impurity derived from unreacted monofunctional monomer, multifunctional monomer, chain transfer agent and initiator comprises 0.05 to 20 mole % of the copolymer based on the number of moles of monomers. More preferably, the residual material or impurity derived from unreacted monofunctional monomer, multifunctional monomer, chain transfer agent and initiator comprises 0.05 to 10 mole % of the copolymer based on the number of moles of monomers. Most preferably, the residual material or impurity derived from unreacted monofunctional monomer, multifunctional monomer, chain transfer agent and initiator comprises 0.05 to 5 mole % of the copolymer based on the number of moles of monomers

The use of a chain transfer agent and an initiator is preferred. However, some molecules can perform both functions.

Hydrophilic macroinitiators (where the molecular weight of the pre-formed polymer is at least 1000 Daltons) can be prepared from hydrophilic polymers synthesised by RAFT (or MADIX), or where a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Hydrophobic macroinitiators (where the molecular weight of the preformed polymer is at least 1000 Daltons) can be prepared from hydrophobic polymers synthesised by RAFT (or MADIX), or where a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Responsive macroinitiators (where the molecular weight of the preformed polymer is at least 1000 Daltons) can be prepared from responsive polymers synthesised by RAFT (or MADIX), or where a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

The monofunctional monomer may comprise any carbon-carbon unsaturated compound which can be polymerised by an addition polymerisation mechanism, for example vinyl and allyl compounds. The dispersing power of the branched polymer dispersant, the ratio and type of anchoring, solubilising or stabilising units can be controlled through the choice of monofunctional monomer. The monofunctional monomer may be hydrophilic, hydrophobic, amphiphilic, anionic, cationic, neutral or zwitterionic in nature. The monofunctional monomer may be selected from but not limited to monomers such as: vinyl acids, vinyl acid esters, vinyl aryl compounds, vinyl acid anhydrides, vinyl amides, vinyl ethers, vinyl amines, vinyl aryl amines, vinyl nitrites, vinyl ketones, and derivatives of the aforementioned compounds as well as corresponding allyl variants thereof.

Other suitable monofunctional monomers include: hydroxyl-containing monomers and monomers which can be post-reacted to form hydroxyl groups, acid-containing or acid-functional monomers, zwitterionic monomers and quaternised amino monomers. Oligomeric, polymeric and di- or multi-functionalised monomers may also be used, especially oligomeric or polymeric (meth)acrylic acid esters such as mono(alk/aryl) (meth)acrylic acid esters of polyalkyleneglycol or polydimethylsiloxane or any other mono-vinyl or allyl adduct of a low molecular weight oligomer. Mixtures of more than one monomer may also be used to give statistical, graft, gradient or alternating copolymers.

Vinyl acids and derivatives thereof include: (meth)acrylic acid, fumaric acid, maleic acid, itaconic acid and acid halides thereof such as (meth)acryloyl chloride. Vinyl acid esters and derivatives thereof include: C₁ to C₂₀ alkyl(meth)acrylates (linear and branched) such as for example methyl(meth)acrylate, stearyl(meth)acrylate and 2-ethyl hexyl(meth)acrylate; aryl(meth)acrylates such as for example benzyl(meth)acrylate; tri(alkyloxy)silylalkyl(meth)acrylates such as trimethoxysilylpropyl(meth)acrylate; and activated esters of (meth)acrylic acid such as N-hydroxysuccinamido(meth)acrylate. Vinyl aryl compounds and derivatives thereof include: styrene, aectoxystyrene, styrene sulfonic acid, 2- and 4-vinyl pyridine, vinylbenzyl chloride and vinyl benzoic acid. Vinyl acid anhydrides and derivatives thereof include: maleic anhydride. Vinyl amides and derivatives thereof include: (meth)acrylamide, N-(2-hydroxypropyl)methacrylamide, N-vinyl pyrrolidone, N-vinyl formamide, (meth)acrylamidopropyl trimethyl ammonium chloride, [3-((meth)acrylamido)propyl]dimethyl ammonium chloride, 3-[N-(3-(meth)acrylamidopropyl)-N,N-dimethyl]aminopropane sulfonate, methyl(meth)acrylamidoglycolate methyl ether and N-isopropyl(meth)acrylamide. Vinyl ethers and derivatives thereof include: methyl vinyl ether. Vinyl amines and derivatives thereof include: dimethylaminoethyl(meth)acrylate, diethylaminoethyl(meth)acrylate, diisopropylaminoethyl(meth)acrylate, mono-t-butylaminoethyl (meth)acrylate, morpholinoethyl(meth)acrylate and monomers which can be post-reacted to form amine groups, such as N-vinyl formamide. Vinyl aryl amities and derivatives thereof include: vinyl aniline, 2 and 4-vinyl pyridine, N-vinyl carbazole and vinyl imidazole. Vinyl nitriles and derivatives thereof include: (meth)acrylonitrile. Vinyl ketones and derivatives thereof including acreolin.

Hydroxyl-containing monomers include: vinyl hydroxyl monomers such as hydroxyethyl(meth)acrylate, 1- and 2-hydroxy propyl(meth)acrylate, glycerol mono(meth)acrylate and sugar mono(meth)acrylates such as glucose mono(meth)acrylate. Monomers which can be post-reacted to form hydroxyl groups include: vinyl acetate, acetoxystyrene and glycidyl(meth)acrylate. Acid-containing or acid functional monomers include: (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, mono-2-((meth)acryloyloxy)ethyl succinate and ammonium sulfatoethyl(meth)acrylate. Zwitterionic monomers include: (meth)acryloyl oxyethylphosphoryl choline and betaines, such as [2-((meth)acryloyloxy)ethyl]dimethyl-(3-sulfopropyl)ammonium hydroxide. Quaternised amino monomers include: (meth)acryloyloxyethyltri-(alk/aryl)ammonium halides such as (meth)acryloyloxyethyltrimethyl ammonium chloride.

Oligomeric and polymeric monomers include: oligomeric and polymeric (meth)acrylic acid esters such as mono(alk/aryl)oxypolyalkyleneglycol(meth)acrylates and mono(alk/aryl)oxypolydimethyl-siloxane(meth)acrylates. These esters include for example: monomethoxy oligo(ethyleneglycol)mono(meth)acrylate, monomethoxy oligo(propyleneglycol)mono(meth)acrylate, monohydroxy oligo(ethyleneglycol) mono(meth)acrylate, monohydroxy oligo(propyleneglycol)mono(meth)acrylate, monomethoxy poly(ethyleneglycol)mono(meth)acrylate, monomethoxy poly(propyleneglycol)mono(meth)acrylate, monohydroxy poly(ethyleneglycol) mono(meth)acrylate and monohydroxy poly(propyleneglycol)mono(meth)acrylate. Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living, polymerisation technique such as poly(1,4-butadiene).

The corresponding allyl monomers to those listed above can also be used where appropriate.

Examples of preferred monofunctional monomers include:

Amide-containing monomers such as (meth)acrylamide, N-(2-hydroxypropyl)methacrylamide, N,N′-dimethyl(meth)acrylamide, N and/or N′-di(alkyl or aryl)(meth)acrylamide, N-vinyl pyrrolidone, [3-((meth)acrylamido)propyl]trimethyl ammonium chloride, 3-(dimethylamino)propyl(meth)acrylamide, 3-[N-(3-(meth)acrylamidopropyl)-N,N-dimethyl]aminopropane sulfonate, methyl (meth)acrylamidoglycolate methyl ether and N-isopropyl(meth)acrylamide; (Meth)acrylic acid and derivatives thereof such as (meth)acrylic acid, (meth)acryloyl chloride (or any halide), (alkyl/aryl)(meth)acrylate; functionalised oligomeric or polymeric monomers such as monomethoxy oligo(ethyleneglycol)mono(meth)acrylate, monomethoxy oligo(propyleneglycol)mono(meth)acrylate, monohydroxy oligo(ethyleneglycol) mono(meth)acrylate, monohydroxy oligo(propyleneglycol)mono(meth)acrylate, monomethoxy poly(ethyleneglycol)mono(meth)acrylate, monomethoxy poly(propyleneglycol)mono(meth)acrylate, monohydroxy poly(ethyleneglycol) mono(meth)acrylate, monohydroxy poly(propyleneglycol)mono(meth)acrylate, glycerol mono(meth)acrylate and sugar mono(meth)acrylates such as glucose mono(meth)acrylate;

vinyl amines such as aminoethyl(meth)acrylate, dimethylaminoethyl(meth)acrylate, diethylaminoethyl(meth)acrylate, diisopropylaminoethyl(meth)acrylate, mono-t-butylamino (meth)acrylate, morpholinoethyl(meth)acrylate; vinyl aryl amines such as vinyl aniline, vinyl pyridine, N-vinyl carbazole, vinyl imidazole, and monomers which can be post-reacted to form amine groups, such as vinyl formamide;

vinyl aryl monomers such as styrene, vinyl benzyl chloride, vinyl toluene, α-methyl styrene, styrene sulfonic acid, vinyl naphthalene and vinyl benzoic acid; vinyl hydroxyl monomers such as hydroxyethyl(meth)acrylate, hydroxy propyl (meth)acrylate, glycerol mono(meth)acrylate or monomers which can be post-functionalised into hydroxyl groups such as vinyl acetate, acetoxy styrene and glycidyl(meth)acrylate;

acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate or acid anhydrides such as maleic anhydride;

zwitterionic monomers such as (meth)acryloyl oxyethylphosphoryl choline and betaine-containing monomers, such as [2-((meth)acryloyloxy)ethyl]dimethyl-(3-sulfopropyl)ammonium hydroxide;

quaternised amino monomers such as (meth)acryloyloxyethyltrimethyl ammonium chloride.

The corresponding allyl monomer, where applicable, can also be used in each case.

Functional monomers, that is monomers with reactive pendant groups which can be post or pre-modified with another moiety following polymerisation can also be used such as for example glycidyl(meth)acrylate, tri(alkoxy)silylalkyl(meth)acrylates such as trimethoxysilylpropyl(meth)acrylate, (meth)acryloyl chloride, maleic anhydride, hydroxyalkyl(meth)acrylates, (meth)acrylic acid, vinylbenzyl chloride, activated esters of (meth)acrylic acid such as N-hydroxysuccinamido(meth)acrylate and acetoxystyrene.

Macromonomers (monomers having a molecular weight of at least 1000 Daltons) are generally formed by linking a polymerisable moiety, such as a vinyl or allyl group, to a pre-formed monofunctional polymer via a suitable linking unit such as an ester, an amide or an ether. Examples of suitable polymers include: mono functional poly(alkylene oxides) such as monomethoxy[poly(ethyleneglycol)] or monomethoxy[poly(propyleneglycol)], silicones such as poly(dimethylsiloxane)s, polymers thrilled by ring-opening polymerisation such as poly(caprolactone) or poly(caprolactam) or mono-functional polymers formed via living polymerisation such as poly(1,4-butadiene).

Preferred macromonomers include: monomethoxy- or hydroxyl-[poly(ethyleneglycol)]mono(methacrylate), monomethoxy- or hydroxyl-[poly(propyleneglycol)]mono(methacrylate) and mono(meth)acryloxypropyl-terminated poly(dimethylsiloxane).

When the monofunctional monomer is providing the necessary hydrophilicity in the copolymer, it is preferred that the monofunctional monomer is a residue of a hydrophilic monofunctional monomer, preferably having a molecular weight of at least 1000 Daltons.

Hydrophilic monofunctional monomers include: (meth)acryloyl chloride, N-hydroxysuccinamido (meth)acrylate, styrene sulfonic acid, maleic anhydride, (meth)acrylamide, N-(2-hydroxypropyl)methacrylamide, N-vinyl pyrrolidinone, N-vinyl formamide, quaternised amino monomers such as (meth)acrylamidopropyl trimethyl ammonium chloride, [3-((meth)acrylamido)propyl]trimethyl ammonium chloride and (meth)acryloyloxyethyltrimethyl ammonium chloride, 3-[N-(3-(meth)acrylamidopropyl)-N,N-dimethyl]aminopropane sulfonate, methyl(meth)acrylamidoglycolate methyl ether, glycerol mono(meth)acrylate, monomethoxy and monohydroxy oligo(ethylene oxide) (meth)acrylate, sugar mono(meth)acrylates such as glucose mono(meth)acrylate, (meth)acrylic acid, vinyl phosphonic acid, fumaric acid, itaconic acid, 2-(meth)acrylamido 2-ethyl propanesulfonic acid, mono-2-((meth)acryloyloxy)ethyl succinate, ammonium sulfatoethyl(meth)acrylate, (meth)acryloyl oxyethylphosphoryl choline and betaine-containing monomers such as [2-((meth)acryloyloxy)ethyl]dimethyl-(3-sulfopropyl)ammonium hydroxide. Hydrophilic macromonomers may also be used and include: monomethoxy and monohydroxy polyethylene oxide) (meth)acrylate and other hydrophilic polymers with terminal functional groups which can be post-functionalised with a polymerisable moiety such as (meth)acrylate, (meth)acrylamide or styrenic groups.

Hydrophobic monofunctional monomers include: C1 to C28 alkyl(meth)acrylates (linear and branched and (meth)acrylamides, such as methyl(meth)acrylate and stearyl (meth)acrylate, aryl(meth)acrylates such as benzyl(meth)acrylate, tri(alkyloxy)silylalkyl(meth)acrylates such as trimethoxysilylpropyl(meth)acrylate, styrene, acetoxystyrene, vinylbenzyl chloride, methyl vinyl ether, vinyl formamide, (meth)acrylonitrile, acreolin, 1- and 2-hydroxy propyl(meth)acrylate, vinyl acetate, 5-vinyl 2-norbornene, Isobornyl methacrylate and glycidyl(meth)acrylate. Hydrophobic macromonomers may also be used and include: monomethoxy and monohydroxy poly(butylene oxide) (meth)acrylate and other hydrophobic polymers with terminal functional groups which can be post-functionalised with a polymerisable moiety such as (meth)acrylate, (meth)acrylamide or styrenic groups.

Responsive monofunctional monomers include: (meth)acrylic acid, 2- and 4-vinyl pyridine, vinyl benzoic acid, N-isopropyl(meth)acrylamide, tertiary amine(meth)acrylates and (meth)acrylamides such as 2-(dimethyl)aminoethyl(meth)acrylate, 2-(diethylamino)ethyl (meth)acrylate, diisopropylaminoethyl(meth)acrylate, mono-t-butylaminoethyl (meth)acrylate and N-morpholinoethyl(meth)acrylate, vinyl aniline, 2- and 4-vinyl pyridine, N-vinyl carbazole, vinyl imidazole, hydroxyethyl(meth)acrylate, hydroxypropyl (meth)acrylate, maleic acid, fumaric acid, itaconic acid and vinyl benzoic acid. Responsive macromonomers may also be used and include: monomethoxy and monohydroxy poly(propylene oxide) (meth)acrylate and other responsive polymers with terminal functional groups which can be post-functionalised with a polymerisable moiety such as (meth)acrylate, (meth)acrylamide or styrenic groups.

The multifunctional monomer or brancher may comprise a molecule containing at least two vinyl groups which may be polymerised via addition polymerisation. The molecule may be hydrophilic, hydrophobic, amphiphilic, neutral, cationic, zwitterionic, oligomeric or polymeric. Such molecules are often known as cross-linking agents in the literature and may be prepared by reacting any di- or multifunctional molecule with a suitably reactive monomer. Examples include: di- or multivinyl esters, di- or multivinyl amides, di- or multivinyl aryl compounds, di- or multivinyl alk/aryl ethers. Typically, in the case of oligomeric or polymeric di- or multifunctional branching agents, a linking reaction is used to attach a polymerisable moiety to a di- or multifunctional oligomer or polymer. The brancher may itself have more than one branching point, such as T-shaped divinylic oligomers or polymers. In some cases, more than one multifunctional monomer may be used. When the multifunctional monomer is providing the necessary hydrophilicity in the copolymer, it is preferred that the multifunctional monomer has a molecular weight of at least 1000 Daltons.

The corresponding allyl monomers to those listed above can also be used where appropriate.

Preferred multifunctional monomers include but are not limited to: divinyl aryl monomers such as divinyl benzene; (meth)acrylate diesters such as ethylene glycol di(meth)acrylate, propyleneglycol di(meth)acrylate and 1,3-butylenedi(meth)acrylate; polyalkylene oxide di(meth)acrylates such as tetraethyleneglycol di(meth)acrylate, poly(ethyleneglycol)di(meth)acrylate and poly(propyleneglycol)di(meth)acrylate; divinyl (meth)acrylamides such as methylene bisacrylamide; silicone-containing divinyl esters or amides such as (meth)acryloxypropyl-terminated poly(dimethylsiloxane); divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate. Further examples include vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene).

Macrocrosslinkers or macrobranchers (multifunctional monomers having a molecular weight of at least 1000 Daltons) are generally formed by linking a polymerisable moiety, such as a vinyl or aryl group, to a pre-formed multifunctional polymer via a suitable linking unit such as an ester, an amide or an ether. Examples of suitable polymers include: di-functional poly(alkylene oxides) such as poly(ethyleneglycol) or poly(propyleneglycol), silicones such as poly(dimethylsiloxane)s, polymers formed by ring-opening polymerisation such as poly(caprolactone) or poly(caprolactam) or poly-functional polymers formed via living polymerisation such as poly(1,4-butadiene).

Preferred macrobranchers include: poly(ethyleneglycol)di(meth)acrylate, poly(propyleneglycol)di(meth)acrylate, methacryloxypropyl-terminated poly(dimethylsiloxane), poly(caprolactone)di(meth)acrylate and poly(caprolactam) di(meth)acrylamide.

Branchers include: methylene bisacrylamide, glycerol di(meth)acrylate, glucose di- and tri(meth)acrylate, oligo(caprolactam) and oligo(caprolactone). Multi end-functionalised hydrophilic polymers may also be functionalised using a suitable polymerisable moiety such as a (meth)acrylate, (meth)acrylamide or styrenic group.

Further branchers include: divinyl benzene, (meth)acrylate esters such as ethyleneglycol di(meth)acrylate, propyleneglycol di(meth)acrylate and 1,3-butylene di(meth)acrylate, oligo(ethylene glycol)di(meth)acrylates such as tetraethylene glycol di(meth)acrylate, tetra- or tri-(meth)acrylate esters such as pentaerthyritol tetra(meth)acrylate, trimethylolpropane tri(meth)acrylate and glucose penta(meth)acrylate. Multi end-functionalised hydrophobic polymers may also be functionalised using a suitable polymerisable moiety such as a (meth)acrylate, (meth)acrylamide or styrenic group.

Multifunctional responsive polymers may also be functionalised using a suitable polymerisable moiety such as a (meth)acrylate, (meth)acrylamide or styrenic group such as poly(propylene oxide)di(meth)acrylate.

### EXAMPLES

The present invention will now be explained in more detail by reference to the following non-limiting examples.

In the following examples, copolymers are described using the following nomenclature:

### (Monomer G)_{g}(Monomer J)ⱼ(Brancher L)ₗ(Chain Transfer Agent)_{d}

wherein the values in subscript are the molar ratios of each constituent normalised to give the monofunctional monomer values as 100, that is, g+j=100. The degree of branching or branching level is denoted by l and d refers to the molar ratio of the chain transfer agent.

For example: Methacrylic acid₁₀₀Ethyleneglycol dimethacrylate₁₅Dodecane thiol₁₅ would describe a polymer containing methacrylic acid:ethyleneglycol dimethacrylate:dodecane thiol at a molar ratio of 100:15:15.

### ABBREVIATIONS

### Monomers:

AA—Acrylic acid,
DMA—2-dimethylaminoethyl methacrylate,
LMA—Lauryl methacrylate,
PEGMA—Poly(ethylene glycol) methacrylate 1000 Da,
PEG2kMA—Poly(ethylene glycol) methacrylate 2000 Da

### St—Styrene,

VP—4-Vinyl pyridine,

### Branchers:

DVB—Divinyl benzene.
EGDMA—Ethyleneglycol dimethacrylate
TEGDMA—Triethylene glycol methacrylate

### Chain Transfer Agent CTA

### DDT—Dodecanethiol.

2,4-DMP—2,4-diphenyl-4-methyl-1-pentene
3-MPA—3-Mercaptopropionic acid

### TG—Thioglycerol

### Initiators

### AIBN—2,2′-Azobisisobutyronitrile.

TBPO—di-tert Butyl peroxide,

### V-88—Vazo 88, 1,1′-Azobis(Cyclohexanecarbonitrile)

### Solvents

### MeOH—Methanol

MPA—1-Methoxy-2-propyl acetate.
PGDA—Propyleneglycol diacetate

### THF—Tetrahydrofuran

General synthetic procedure for polymeric materials in Table 1. The monomers, brancher, chain transfer agent, initiator and solvent were added to a class vessel fitted with an overhead stirrer. The vessel was sealed and degassed by bubbling nitrogen through the solution for between 30 to 60 minutes. The vessel was then heated to the set temperature with constant agitation, for 17 hours. The resulting polymer solution was then either used without purification or alternatively the polymer was precipitated into a non-solvent isolated by filtration and dried.

GPC procedure. Triple Detection-Size Exclusion Chromatography was performed on a Viscotek triple detection instrument. The columns used were two ViscoGel HHR-H columns and a guard column with an exclusion limit for polystyrene of 10⁷ g·mol⁻¹. Tetrahydrofuran (THF) was the mobile phase, the column oven temperature was set to 35° C., and the flow rate was 1 mL·min⁻¹. The samples were prepared for injection by dissolving 10 mg of polymer in 1.0 mL of HPLC grade THF and filtered using an Acrodisc® 0.2 μm PTFE membrane. 0.1 mL of this mixture was then injected, and the data was collected for 30 minutes. Omnisec was used to collect and process the signals transmitted from the detectors to the computer and to calculate the molecular weight of the polymers.

NMR procedure. Triple Detection-Size Exclusion Chromatography was performed on a Viscotek triple detection instrument. The columns used were two ViscoGel HHR-H columns and a guard column with an exclusion limit for polystyrene or 10⁷ g·mol⁻¹.

THF was the mobile phase, the column oven temperature was set to 35° C., and the flow rate was 1 mL·min⁻¹. The samples were prepared for injection by dissolving 10 mg of polymer in 1.5 mL of HPLC grade THF and filtered using an Acrodisc® 0.2 μm PTFE membrane. 0.1 mL of this mixture was then injected, and data was collected for 30 minutes. Omnisec was used to collect and process the signals transmitted from the detectors to the computer and to calculate the molecular weight.

### General Example 1

### Branched poly(4-vinyl pyridine-co-styrene-co-ethyleneglycol dimethacrylate)

VP₂₅ST₇₅ECDMA₁₀DDT₁₅. Into a vessel were placed Styrene (15.2 g, 145.5 mmol), 4-vinyl pyridine (5.1 g, 48.5 mmol), ethylene glycol dimethacrylate (3.8 g, 19.4 mmol), dodecane thiol (5.89 g, 29.1 mmol) and di-tertiary butyl peroxide (0.48 mL, 2.6 mmol) were dissolved in propylene glycol diacetate (70 g). The vessel was sealed and the solution degassed with nitrogen for one hour with constant agitation. The mixture was then heated to 130° C., for 17 hours, to give a yellow solution which showed greater than 99% monomer conversion by ¹H NMR. The polymer was then either used directly from the reaction solution or isolated in a solid form by precipitating into light petroleum ether, isolation by filtration and drying under vacuum, at 40° C., to give a 75% recovered yield.

GPC. Mn: 13,000; Mw: 81,600; Eluent: THF

Rheology measurement procedure. All solutions were measured using a Bohlin CVO 120 controlled stress rheometer fitted with a CP2°/52 mm cone. Mill base solutions were measured at 25° C. and the viscosity was recorded with increasing shear rate of 0.4 to 1000 s⁻¹. The let-down solutions were measured at 25° C. and with a fixed shear rate of 600 s⁻¹.

Procedure for measuring particle-size. A glass cuvette was filled to three quavers of its total volume with an appropriate solvent. To this solvent was added six pasteur pipette drops of let-down solution and the contents were then mixed. The glass cuvette was inserted into a Malvern Zetasizer Nano and two measurements were taken, at 20° C., of the d-average particle size.

Pigment Dispersion Procedure. A mixture of varying diameter stainless steel balls (300 g of 6 mm diameter, 250 g of 5 mm diameter and 230 g of 4 mm diameter) were added to a 250 mL steel container. The container was then charged with 20 g of pigment, and a solution of dispersant as stated in Table 2 and 3.

The steel container was then sealed and rolled on a mechanical roller at 33 rpm for 24 hours. Following the milling stage the mill base viscosity was measured at 1 and 400 s⁻¹. The dispersant was then diluted with solvent to give a dispersion with a concentration of pigment of 3% w/w, the viscosity of this diluted dispersion was also measured together with the particle size. The dispersant solution was then gently agitated prior to dosing into the graduated tubes and placing in an incubator for the set period of time. The tubes were then incubated at 50° C. for 7 days. Once more the solution viscosity was recorded and compared to the pre-incubated value, the stability of the dispersion was determined by noting the amount of clear solution (clarity) in the tube.

Where the pigments were prepared in water a further rub-out test was performed, as described below.

General procedure for measuring ΔE values. A jar was charged with 0.8 g of a water-based pigment dispersion (containing 20% w/w pigment) and 40 g of Dulux Vinyl Matt. Once mixed, a 100 micron wire wound applicator rod was used to apply an even film of the paint to a white card (approx 10×15 cm). The coating was left for one minute to dry, and then a section was rubbed in a circular motion, by finger, twenty times. The coating was then left overnight to dry. The ΔE value was determined by using a Konica Minolta Spectrophotometer CM-2300d to measure the rubbed and un-rubbed area of the coating.

Dispersion of Copper Phthalocyanine at Room Temperature. The milling procedure described previously was performed utilising the synthesised, branched polymer dispersants, in addition two control experiments were performed where no dispersants were used and the pigments were milled with solvent only. Where a polymeric dispersant was used the level was 3% by weight copper Phthalocyanine to 3% (dry) by weight branched polymer dispersant, that is, a total solids content of 6% in PGDA. When the milling was completed the dispersions were Idled into a 25 mL graduated tube and left undisturbed at room temperature for 21 days.

Example 1 showed a dispersant stability of greater than 21 days at room temperature with no sedimentation of the dispersant solution

Accelerated Stability Testing for Phthalocyanine Dispersions. In order to assess the stability of the phthalocyanine dispersions in PGDA a series of accelerated stability tests were performed where the dilute milled dispersions were incubated in an oven at 54° C.

In addition to the milling procedure described previously a concentrated milling procedure was also employed where 20 g of pigment, a quantity of dispersant and PGDA was milled as before. Following the grinding stage the dispersant was diluted down with PGDA to give a dispersion with a concentration of pigment of 3% w/w, the dispersant solution was then gently agitated prior to dosing into the graduated tubes and placing in the incubator for the set period of time.

Example 1 showed a dispersant stability of greater than 77 days at 54° C. with no sedimentation of the dispersant solution for the diluted mill-base concentrate.

The following examples were prepared via the described experimental procedure and their molecular weights were determined via triple detection gel permeation chromatography.

TABLE 1
Compositions and characterisation of branched addition polymers

| | | | | | | Initiator | |
|---|---|---|---|---|---|---|---|
| | | | | Reaction | | (% to | |
| | | Solvent | Solids | temp. | Initiator | vinyl | |
| Ref. | Polymer Description | Used | (%) | (° C.) | type | group) | Mw/Da |
| 1 | VP₅₀ST₅₀EGDMA₁₀DDT₁₅ | PGDA | 30 | 70 | AIBN | 2.26 | 35 800 |
| 2 | VP₈₅ LMA₁₅ EGDMA₁₀ | PGDA | 30 | 130 | TBPO | 1.15 | 12 800 |
| | DDT₁₅ | | | | | | |
| 3 | VP₂₅ST₇₅EGDMA₁₀DDT₁₅ | Toluene | 40 | 140 | V-88 | 1.12 | 20 600 |
| 4 | ST₁₀₀EGDMA₁₀DDT₁₅ | PGDA | 30 | 140 | TBPO | 1.12 | 20 800 |
| 5 | DMA₂₅ST₇₅EGDMA₁₀DDT₁₁ | MPA | 40 | 140 | TBPO | 1.19 | 11 300 |
| 6 | AA₂₅ST₇₅EGDMA₁₀DDT₁₅ | Toluene | 40 | 140 | V-88 | 0.91 | 34 000 |
| 7 | DMA₁₀₀EGDMA₁₀DDT₁₅ | PGDA | 30 | 70 | AIBN | 1.35 | 17000 |
| 8 | AA_{97.5}PEG2KMA_{2.5}EGDMA₁₅TG₁₅ | PGDA | 29 | 70 | AIBN | 0.75 | 48 400 |

The dispersants were then formulated with different pigments in two different solvent systems, dipropyleneglycol diacetate and dipropylenedlycol diacrylate and compared against a linear styrenic polymer LP-1 and two commercial comparative dispersants CCE-1 and CCE-2.

TABLE 2
Dispersions prepared in dipropyleneglycol diacetate

| | Dispersant | | Millbase | | Formulated Let-Down | | |
|---|---|---|---|---|---|---|---|
| | Dose (% | | Viscosity (cP) | | Solution Viscosity at | Visosity | Clarity |
| Ref | w/w) | Pigment | at 1 s⁻¹ | at 400 s⁻¹ | 600 s⁻¹ (cP) | Change (%) | (%) |
| CCE-1 | 20 | Monarch | 14700 | 162 | 3.6 | 11 | 0 |
| | | Black 800 | | | | | |
| 3 | 20 | Monarch | 8 900 | 4 | 3.7 | 15 | 0 |
| | | Black 800 | | | | | |
| 3 | 2 | Monarch | 2 900 | 36 | 4.9 | 31 | 0 |
| | | Black 800 | | | | | |
| 3 | 20 | Heuco | 3 600 | 140 | 4.7 | 9 | 0 |
| | | Green | | | | | |
| 3 | 20 | Textile | 1 500 | NM | 3.8 | 0 | 0 |
| | | Black | | | | | |
| 4 | 2 | Monarch | 4 400 | 35 | 5.2 | 15 | 0 |
| | | Black 800 | | | | | |

CCE-1 refers to commercial comparative example 1

TABLE 3
Dispersions prepared in dipropyleneglycol diacrylate

| | | | | | Formulated | | | |
|---|---|---|---|---|---|---|---|---|
| | | | | | Let-Down | | | |
| | Dispersant | | Millbase | | Solution | | | Particle |
| | Dose (% | | Viscosity (cP) | | Viscosity at | Visosity | | Size |
| Ref | w/w) | Pigment | at 1 s⁻¹ | at 400 s⁻¹ | 600 s⁻¹ (cP) | Change (%) | Clarity (%) | (nm) |
| LP-1 | 8 | Special | 10400 | 235 | 22.2 | 5 | 1 | 15100 |
| | | black 250 | | | | | | |
| CCE-2 | 0.8 | Special | 18800 | 215 | 21.9 | 7.9 | 24 | 2700 |
| | | black 250 | | | | | | |
| 5 | 4.00 | Special | 5 500 | 167 | 20.8 | 19 | 0 | NM |
| | | Black 250 | | | | | | |
| 5 | 1.60 | Special | 5 000 | 133 | 18.6 | 23 | 37 | 1 900 |
| | | Black 250 | | | | | | |
| 5 | 0.80 | Special | 5 300 | 93 | 17 | NM | 31 | 2 300 |
| | | Black 250 | | | | | | |
| 5 | 0.16 | Special | 9 400 | 112 | 17.4 | NM | 33 | 2 500 |
| | | Black 250 | | | | | | |
| 5 | 15.40 | Irgalite | 7 700 | 251 | 24.5 | 11 | 33 | 4 800 |
| | | Blue | | | | | | |
| | | GLO (15:3) | | | | | | |
| 5 | 7.50 | Irgalite | 5 700 | 197 | 17 | NM | 43 | 2 300 |
| | | Blue | | | | | | |
| | | GLO (15:3) | | | | | | |
| 5 | 2.20 | Irgalite | 4 100 | 141 | 16.0 | NM | 31 | 2 300 |
| | | Blue | | | | | | |
| | | GLO (15:3) | | | | | | |
| 5 | 1.10 | Irgalite | 5 300 | 109 | 16.6 | NM | 17 | 2 200 |
| | | Blue | | | | | | |
| | | GLO (15:3) | | | | | | |

LP-1 refers to comparative linear example polymer
CCE-2 refers to commercial comparative example-2
NM—not measured

TABLE 4
Results obtained for dispersions prepared in water.

| | Dispersant | | Millbase | | |
|---|---|---|---|---|---|
| | Dose | | Viscosity (cP) | | Delta |
| Ref | (% w/w) | Pigment | at 1 s⁻¹ | at 400 s⁻¹ | E |
| CCE -3 | 5.0 | Monarch Black 800 | 3 500 | 73 | 1.1 |
| 8 | 5.0 | Monarch Black 800 | 3 200 | 40 | 2.7 |
| 7 | 5.0 | Lamp Black | 219 | 7 | 0.2 |
| 8 | 4.7 | Sicotrans Rot L2817 | 408 | 45 | 2.8 |
| | | (FeO) BASF | | | |
| 8 | 2.0 | Monolite Blue (15:3) | 108 | 10 | 3.6 |
| 8 | 2.7 | Monolite Blue (15:3) | 17 | 7 | 2.7 |
| 7 | 2.7 | Monolite Blue (15:3) | 4 | 3 | 4.6 |
| 7 | 2.0 | Monolite Blue (15:3) | 3 700 | 41 | 7.5 |
| 7 | 2.7 | Heuco Red | 6 | 4 | 5.0 |
| CCE-4 | 2.7 | Heuco Red | 20 | 14 | 3.1 |

CCE-3 and CCE-4 refer to commercial comparative examples 3 and 4 respectively.

As the data shows the branched polymer examples provided a lower low shear viscosity than the commercial materials indicating their high dispersing power.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations. The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The inclusion or discussion of a reference is not an admission that it is prior art to the present invention, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

What is claimed is:

1. A method of using of a branched addition copolymer as a dispersant in a gaseous, liquid or solid formulation wherein the copolymer is obtainable by an addition polymerisation process, wherein said copolymer comprises:
at least two chains which are covalently linked by a bridge other than at their ends; and wherein the at least two chains comprise at least one ethylenically monounsaturated monomer, and wherein the bridge comprises at least one ethylenically polyunsaturated monomer; and wherein
the polymer comprises a residue of a chain transfer agent; and wherein
the mole ratio of polyunsaturated monomer(s) to monounsaturated monomer(s) is in a range of from 1:100 to 1:4; and wherein
the brandied copolymer dispersant contains anchoring; solubilising or stabilising moieties and wherein the resulting copolymer has a weight average molecular weight of less than 100,000 Da.

2. The method of claim 1 wherein the polymer comprises a residue of a chain transfer agent and a residue of an initiator.

3. The method of claim 1 wherein the branched copolymer dispersant is used to stabilise solid particles within a liquid phase to form a stable dispersion.

4. The method of claim 1 wherein the branched copolymer dispersant is used to stabilise solid particles within a solid phase to form a stable dispersion.

5. The method of claim 1 wherein the branched copolymer dispersant is used to stabilise solid particles within a gaseous phase to form a stable dispersion.

6. The method of claim 3 wherein the solid particles to be stabilised are particles in a hydrophobic or hydrophilic liquid.

7. The method of claim 1 wherein the copolymer has a weight average molecular weight of 2,000 Da to 1,000,000 Da.

8. The method of claim 1 wherein the copolymer has a weight average molecular weight of 2,000 Da to 50,000 Da.

9. The method of claim 1 wherein the branched copolymer is used as dispersants for pigments.

10. The method of claim 1 wherein the branched copolymer is used as dispersants for metal salts and metallic particles.

11. The method of claim 1 wherein the branched copolymer is used as dispersants for cement and/or powder coatings.

12. The method of claim 1 wherein the branched copolymer is used as dispersants for lubricating media.

13. The method of claim 1 wherein the branched copolymer is used as dispersants for organic molecules in the pharmaceutical, agrochemical, biocides, food colorants, flavourings and fragrances industries.

14. The method of claim 1 wherein when a composition of the polymer is applied to a dispersion the ratio of the dispersed phase to polymer is in the range 0.1:1 to 1000:1.

15. The method of claim 1 wherein when a composition of the polymer is applied to a dispersion the ratio of the dispersed phase to polymer is in the range of 0.1:1 to 500:1.

16. The method of claim 1 wherein when a composition of the polymer is applied to a dispersion the ratio of the dispersed phase to polymer is in the range of 0.2:1 to 200:1.

17. The method of claim 1 wherein the branched copolymers used as dispersants are branched, non-cross-linked addition polymers.

18. The method of claim 1 wherein the residue of the chain transfer agents comprises 0.05 to 80 mole % of the copolymer based on the number of moles of monofunctional monomer.

19. The method of claim 1 wherein the residue of the chain transfer agents comprises 0.05 to 30 mole % of the copolymer based on the number of moles of monofunctional monomer.

20. The method of claim 1 wherein the residue of the initiator comprises 0 to 10% w/w of the copolymer based on the total weight of the monomers.

21. The method of claim 1 wherein the residue of the initiator comprises 0.001 to 5% w/w of the copolymer based on the total weight of the monomers.

22. The method of claim 1 wherein the residue of the initiator comprises 0.001 to 3% w/w of the copolymer based on the total weight of the monomers.

23. The method of claim 1 wherein the monofunctional monomer is selected from the group consisting of:
vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives of the aforementioned compounds as well as corresponding allyl variants thereof.

24. The method of claim 1 wherein the multifunctional monomer or brancher is selected from the group consisting of
divinyl aryl monomers such as divinyl benzene; (meth)acrylate diesters such as ethylene glycol di(meth)acrylate, propyleneglycol di(meth)acrylate and 1,3-butylenedi(meth)acrylate; polyalkylene oxide di(meth)acrylates such as tetraethyleneglycol di(meth)acrylate, polyethyleneglycol)di(meth)acrylate and poly(propyleneglycol) di(meth)acrylate; divinyl(meth)acrylamides such as methylene bisacrylamide; silicone-containing divinyl esters or amides such as (meth)acryloxypropyl-terminated poly(dimethylsiloxane); divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate, vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene)

25. The method of claim 1 wherein at least one of the monounsaturated monomer(s) and polyunsaturated monomer(s) and chain transfer agent(s) is a hydrophilic residue; and
at least one of one of the monounsaturated monomer(s) and polyunsaturated monomer(s) and chain transfer agent(s) is a hydrophobic residue.

26. A branched addition copolymer suitable for use as a dispersant in a gaseous, liquid or solid formulation wherein the copolymer is obtainable by an addition polymerisation process, wherein said copolymer comprises:
at least two chains which are covalently linked by a bridge other than at their ends; and wherein the at least two chains comprise at least one ethylenically monounsaturated monomer; and wherein the bridge comprises at least one ethylenically polyunsaturated monomer; and wherein
the polymer comprises a residue of a chain transfer agent; and wherein
the mole ratio of polyunsaturated monomer(s) to monounsaturated monomer(s) is in a range of from 1:100 to 1:4; and wherein
the branched copolymer dispersant contains anchoring, solubilising or stabilising moieties and wherein the resulting copolymer has a weight average molecular weight of less than 100,000 Da.
